(19) United States
(12) Patent Application Publication
(10) Pub. No.: US 2004/0121406 A1
(43) Pub. Date: Jun. 24, 2004

(54) Methods and formulations for increasing the affinity of a1 adenosine receptor ligands for the a1 adenosine receptor

(76) Inventor: Constance Neely Wilson, Raleigh, NC (US)

Correspondence Address: MYERS BIGEL SIBLEY & SAJOVEC, PO BOX 37428, RALEIGH, NC, 27627, US

(21) Appl. No.: 10/475,925

(22) PCT Filed: May 23, 2002
(86) PCT No.: PCT/US02/16218

(51) Int. Cl. G01N 33/53; A61K 39/395; A61K 31/739

(52) U.S. Cl. 435/7.1; 514/54; 424/144.1

(57) ABSTRACT

Glycolipids are useful for enhancing the affinity of A₁ adenosine receptor ligands for the A₁ adenosine receptor. Glycolipids are accordingly useful in diagnostic and therapeutic methods that require the delivery or administration of A₁ adenosine ligands.

### FIELD OF THE INVENTION

The present invention relates to methods of delivering or administering A₁ adenosine receptor ligands for therapeutic or diagnostic purposes, and methods of enhancing the efficacy of the same.

### BACKGROUND OF THE INVENTION

A₁ adenosine receptors (A₁AR) are involved in a vast number of peripheral and central regulatory mechanisms such as, for example, vasodilation, cardiac depression, inhibition of lipolysis, inhibition of insulin release, potentiation of glucagon release in the pancreas, inhibition of neurotransmitter release from nerve endings, tumorigenesis and development of fibrosis and/or sclerosis. A₁ adenosine receptor ligands (A₁AR ligands) such as A₁ adenosine receptor agonists and antagonists are accordingly useful in numerous diagnostic techniques and therapeutic methods.

The use of A₁ adenosine receptor analogs in diagnostic techniques is set forth in, for example, U.S. patent application Ser. No. 08/652,928, filed May 24, 1996 (now U.S. Pat. No. 5,773,306 to Neely, issued Jun. 30, 1998), U.S. patent application Ser. No. 08/748,559, filed Nov. 8, 1996 (now U.S. Pat. No. 6,159,701 to Neely, issued Dec. 12, 2000, hereinafter the '701 patent), and in U.S. patent application Ser. No. 09/569,394 to Neely, filed May 12, 2000, all of which are incorporated herein in their entirety by reference. Therapeutic uses of A₁ adenosine receptor ligands are set forth in, for example, the '701 patent and in U.S. Pat. No. 09/224,534, filed Dec. 31, 1998 (now U.S. Pat. No. 6,117,445 to Neely, issued Sep. 12, 2000, hereinafter the '445 patent), which is also incorporated herein in its entirety by reference.

Present methods of using A₁ adenosine receptor ligands have certain limitations. For example, many of these ligands lack optimal solubility under in vivo conditions, and may have low potency or lack of selectivity for adenosine receptors. Moreover, in diagnostic techniques utilizing the A₁AR (e.g., competitive binding assays for determining the binding of putative A₁ adenosine receptor ligands, monoclonal antibodies, etc. to the receptor), a recurring problem is the stability of materials that serve as the source for the receptor (e.g., membrane preparations). In order to combat the stability issues of receptor protein source, membranes are often maintained (e.g., plated) in an environment containing lactose and other stabilizers. Even in the presence of these stabilizers, however, the shelf-life/stability of A₁AR preparations is limited. It is desirable to have means for increasing the stability of such preparations in diagnostic methods that utilize the A₁AR.

Additionally, it is advantageous to have means for increasing the efficiency and/or strength (e.g., affinity) of binding of A₁AR ligands for the A₁AR, particularly when such ligands are administered for therapeutic purposes. One desirable result of increasing the affinity of binding of A₁AR ligands to the A₁AR would be an increase in the bioavailability of drugs that are A₁AR ligands to the subject being treated. Another advantage may be the increase of solubility of the A₁AR ligand under in vivo and in situ conditions. Increasing bioavailability and solubility of these ligands would generally decrease the amount of ligand that would have to be administered to the subject.

Selective analogs of adenosine receptor ligands have been developed through the “functionalized congener” approach. For example, analogs of adenosine receptor ligands bearing functionalized chains have been synthesized and attached covalently to various organic moieties such as amines and peptides. Attachment of polar groups to xanthine congeners has been found to increase, for example, water solubility. Jacobson et al. have proposed various derivatives of adenosine and theophylline for use as receptor antagonists. See J. Med. Chem. 35, 408-422 (1992). Jacobson et al. describes the finding that hydrophobic substituents are able to potentially enhance A₁AR ligand affinity for the A₁AR. However, they also report that such substituents may result in a decrease in solubility, thus rendering the antagonists less soluble in vivo.

It has been shown that unsaturated fatty acids in micromolar concentrations induce inhibition of A₁AR in rat brain membranes, while saturated fatty acids, G_{M1}-ganglioside, and lysophospholipids were apparently without effect in A₁AR ligand binding at the same concentrations. See K. Domanska et al., NeuroReport 4, 451-453 (1993). This A₁AR agonist-binding inhibition was non-competitive and did not affect the Kd value. The inhibition was also partially reversible by albumin binding. Polyamines such as spermine have also been shown to modulate the binding of A₁AR agonists to the A₁AR. See R. Wasserkort et al., Neuroscience Letters 124, 183-186 (1991). Jacobson et al. have also disclosed that adenosine analogs with covalently attached lipids have enhanced potency at A₁ARs. FEBS Letters 1,2: 97-102 (1987). However, the use of glycolipids in order to enhance A₁AR ligand binding is not discussed.

### SUMMARY OF THE INVENTION

The present invention relates to the inventor's surprising discovery that glycolipids increase the affinity of A₁ adenosine receptor ligands for the A₁ adenosine receptor. Accordingly, one aspect of the invention is a method of increasing the affinity of an A₁ adenosine receptor (A₁AR) ligand for an A₁ adenosine receptor comprising contacting an A₁ adenosine receptor ligand with a glycolipid or an analog thereof, and binding the A₁ adenosine receptor ligand with an A₁ adenosine receptor.

A second aspect of the invention is a method of increasing the affinity of an A₁ adenosine receptor (A₁AR) ligand for an A₁ adenosine receptor comprising contacting an A₁ adenosine receptor with a glycolipid or an analog thereof, and binding the contacted A₁ adenosine receptor with an A₁ adenosine receptor ligand.

A third aspect of the present invention is a method of increasing the affinity of an A₁AR ligand for an A₁AR, comprising contacting the A₁AR ligand with a glycolipid or analog thereof concurrently with binding the A₁AR ligand and the A₁AR.

A fourth aspect of the present invention is a method of delivering an A₁AR ligand to a cell or cell membrane for the purpose of carrying out a diagnostic test, the improvement consisting of increasing the affinity of an A₁AR ligand for the A₁AR by contacting an A₁AR ligand with a glycolipid or an analog thereof and then binding the A₁AR ligand with an A₁AR.

A fifth aspect of the present invention is a method of delivering an A₁AR ligand to a cell or cell membrane for the purpose of carrying out a diagnostic test, the improvement consisting of increasing the affinity of an A₁ adenosine receptor ligand for the A₁ adenosine receptor by contacting an A₁ adenosine receptor with a glycolipid or an analog thereof, and binding the contacted A₁ adenosine receptor with an A₁ adenosine receptor ligand.

A sixth aspect of the present invention is a method of administering an A₁AR ligand to a subject in need of such treatment, the improvement consisting of increasing the affinity of an A₁AR ligand for the A₁AR by administering to the subject the A₁AR ligand with a glycolipid or an analog thereof.

A seventh aspect of the present invention is a method of administering an A₁AR ligand to a subject in need of such treatment, the improvement consisting of increasing the affinity of an A₁AR ligand for the A₁AR by administering to the subject a glycolipid or an analog thereof and then administering an A₁AR ligand.

An eighth aspect of the present invention is a pharmaceutical formulation comprising an A₁ adenosine receptor ligand, a glycolipid or glycolipid analog in an amount sufficient to enhance binding of the A₁ adenosine receptor ligand for the A₁ adenosine receptor, and a pharmaceutically acceptable carrier.

A ninth aspect of the present invention is a method of increasing the affinity of an A₁ adenosine receptor (A₁AR) ligand for a binding site polypeptide of an A₁ adenosine receptor, comprising contacting an A₁ adenosine receptor ligand with a glycolipid or an analog thereof, and binding the contacted A₁ adenosine receptor ligand with a binding site polypeptide of an A₁ adenosine receptor.

A tenth aspect of the present invention is a method of increasing the affinity of an A₁ adenosine receptor (A₁AR) ligand for a binding site polypeptide of an A₁ adenosine receptor, comprising contacting a binding site polypeptide of an A₁ adenosine receptor with a glycolipid or an analog thereof, and binding the contacted binding site polypeptide of an A₁ adenosine receptor with an A₁ adenosine receptor ligand.

In the present invention, A₁ adenosine receptor ligands include but are not limited to A₁ adenosine receptor agonists, A₁ adenosine receptor antagonists, antibodies specific for the A₁ adenosine receptor, and molecules or compounds that are specific for the A₁ adenosine receptor, including but not limited to endotoxin, lipopolysaccharide (LPS), purines, nucleosides, and genetic promoters that are specific for the A₁ adenosine receptor.

The foregoing and other aspects of the present invention are explained in detail in the specification set forth below.

### BRIEF DESCRIPTION OF THE DRAWINGS

FIG. 1 is a graph illustrating competition for the highly selective A₁ adenosine receptor agonist radioligand, [³H] 2-chloro-N⁶-cyclopentyladenosine, [³H] CCPA, binding to human PAEC membranes by Escherichia coli LPS, CCPA, and the selective A₂ adenosine receptor agonist, 2-phenylaminoadenosine (CV 1808). Each data point represents the mean±SEM of three experiments performed on different days and assayed in duplicate.

FIG. 2 is a graph illustrating competition for the highly selective A₂ₐ adenosine receptor agonist radioligand, [³H] CGS 21680, binding to human PAEC membranes by Escherchia coli LPS, the highly selective A₁ adenosine receptor agonist, 2-chloro-N⁶-cyclopentyladenosine (CCPA), and the selective A₂ adenosine receptor agonist, 2-phenylaminoadenosine (CV 1808). Each data point represents the mean±SEM of three experiments performed on different days and assayed in duplicate.

FIG. 3 is a graph illustrating competition for the selective A₁ adenosine receptor antagonist radioligand, [¹²⁵I] BWA844U, binding to human PAEC membranes by LPSs of Escherichia coli, Salmonella typhimurium, Klebsiella pneumoniae, and Pseudomonas aeruginosa. Each data point represents data obtained for one experiment or the average of three different experiments. Each experiment was assayed in duplicate.

FIG. 4 is a graph illustrating the effect of lactocerebroside on binding of the selective A₁ adenosine receptor antagonist radioligand, [¹²⁵I] BWA844U, to human PAEC membranes. Each data point represents data obtained for one experiment or the average of three different experiments. Each experiment was assayed in duplicate.

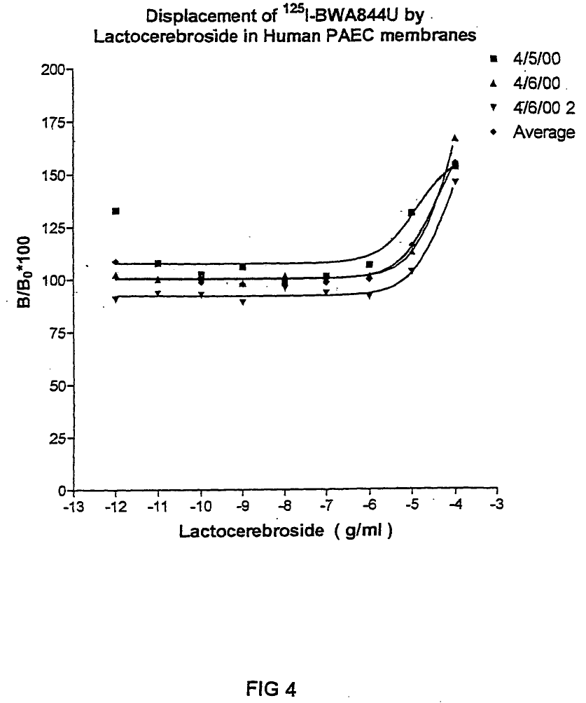

FIG. 5 is a graph illustrating the effect of NBD-galactocerebroside on binding of the selective A₁ adenosine receptor antagonist radioligand, [¹²⁵I] BWA844U, to human PAEC membranes. Each data point represents data obtained for one experiment or the average of three different experiments. Each experiment was assayed in duplicate.

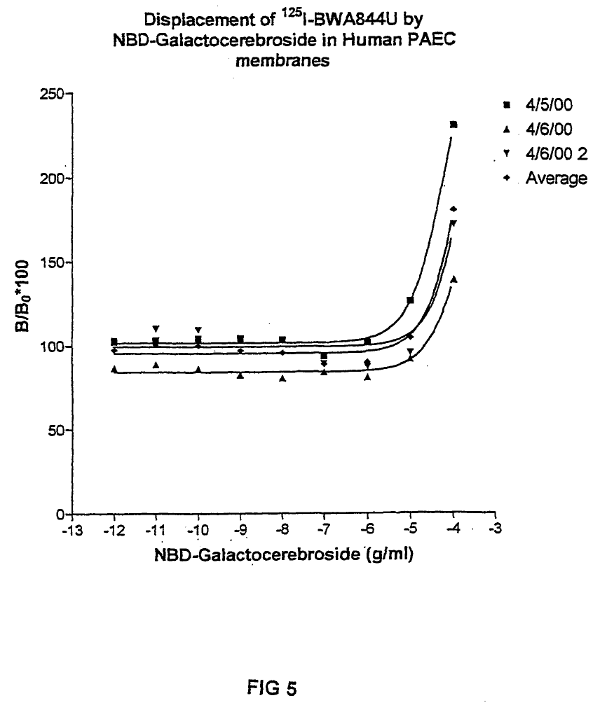

### DETAILED DESCRIPTION OF THE INVENTION

The present invention will now be described with reference to the accompanying figures and specification, in which preferred embodiments of the invention are illustrated. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. All publications, patent applications, patents, and other references mentioned herein are incorporated by reference in their entirety.

The present invention is suitable for both medical and veterinary uses. Suitable subjects include, but are not limited to, mammalian subjects. More preferred subjects are mammalian subjects such as humans, monkeys, pigs, cattle, dogs, horses, cats, sheep, and goats. The most preferred subjects are human subjects.

The present invention relates to the discovery that glycolipids increase the affinity of A₁AR ligands for the A₁AR. In methods utilizing this discovery, A₁AR ligands are delivered or administered with (i.e., in the presence of or bound to) a glycolipid in order to increase the affinity of the ligand for the receptor. Alternatively, the glycolipid is contacted with the receptor, either prior, during or after contact with the ligand. The ligand and the glycolipid (.e., the “active compounds”) may be delivered to a cell or subject concurrently, or may be formulated together (e.g., in a liposome) or drug delivery form, or may be chemically bound, linked or conjugated together.

As used herein, the word “concurrently” means sufficiently close in time to produce a combined (e.g., additive or synergistic) effect. In other words, concurrently may be defined as simultaneously, or it may be defined as two or more events occurring within a short time period of each other.

In one non-limiting embodiment; a glycolipid is administered with the A₁AR ligand according to one or more of several methods. In one embodiment, the glycolipid is chemically linked to (e.g., conjugated, bound to) the A₁AR ligand, for example, via a phenylethylamine or amine group. In another embodiment, the glycolipid is delivered in a liposome that also comprises an A₁AR ligand. In yet another embodiment, the glycolipid is not chemically attached to the ligand but is merely administered concurrently. For example, in a drug delivery method, the glycolipid and the A₁AR ligand may be combined or formulated in the same parenteral injection, or in the same oral drug delivery composition, or in the same aerosol delivery composition. In a formulation according to the present invention, the glycolipid may be formulated with the A₁AR ligand, in, for example, a time release drug delivery form.

In the practice of the present invention, A₁AR ligands include A₁AR antagonists and agonists, as well as other compounds and molecules that have been found to bind to and activate the A₁AR. These compounds and molecules include but are not limited to endotoxin, lipopolysaccharide (LPS), genetic promoters, purines, and nucleosides that bind the A₁AR. See, e.g., Neely, et al., Am. J. Physiol. (Lung Cell Mol. Physiol.) 272, L353 (1997)(endotoxin/LPS); I. Matot et al., Anesth. Analg. 92, 590 (2001) (acadesine [5-amino-4-imidazolecarboxamide riboside], a nucleoside and adenosine-regulating drug that protects the lung from ischemia-reperfusion organ injury). Ligands according to the present invention also include antibodies to the A₁AR, which may be polyclonal antibodies, or may be monoclonal antibodies thereof, or may be active fragments or polypeptides thereof. In preferred embodiments of the invention, the ligands are specific for the A₁AR. In other preferred embodiments, the ligands are highly selective for the A₁AR.

Numerous A₁AR ligands are known and are useful in the practice of the present invention. Certain A₁ adenosine receptor antagonists are set forth in U.S. patent application Ser. No. 08/753,048, filed Nov. 19, 1996 (now U.S. Pat. No. 5,786,360 to Neely, issued Jul. 28,1998), incorporated herein by reference in its entirety. One known class of adenosine receptor antagonists is the xanthine family, which includes caffeine and theophylline. See e.g., Müller et al., J. Med. Chem. 33, 2822-2828 (1990).

A₁AR agonists useful as ligands in the present invention also include but are not limited to adenosine; cyclohexyladenosine; various N⁶-substituted A₁ adenosine agonists including but not limited to N⁶ cyclopentyladenosine, N⁶ R-phenylisopropyladenosine, 2-chloro N⁶ cyclopentyl adenosine (CCPA), N⁶ (p-sulfophenyl) alkyl and N⁶ sulfoalkyl derivatives of adenosine (such as N⁶-(p-sulfophenyl) adenosine; 1-deaza analogues of adenosine including but not limited to N⁶ cyclopentyl 1-2-chloro-1-deaza adenosine (1-deaza-2-Cl—CPA); N⁶ cycloaklyladenosines; N⁶ bicycloalkyladenosines; cycloalkyladenosines; analogs of R—PIA, CHA, and CPA; ribose modified adenosine receptor analogues including but not limited to 3′-deoxy-R—PIA, and combinations and mixtures-thereof. See, e.g., Conti, Naunyn-Schmiedeberg's Arch. Pharmacol. 348:108 (1993); Trivedi, J. Med. Chem. 32:8 (1989); Jacobsen, J. Med. Chem. 35:4143 (1992); Thedford, Expl. Cell. Biol. 57:53 (1989); Trewyn, Exp. Pharmacol. 28:607 (1979); Fleysher, J. Amer. Chem. Soc. (August 1968); Fleysher, J. Amer. Chem. Soc. (November 1969)); Moos, J. Med. Chem. 28:1383 (1985)); (see, e.g., Cristalli, J. Med. Chem. 31:1179 (1988)). Van der Wenden, J. Med. Chem. 38:4000 (1995); Jacobson, PJM Med. Res. Rev. 12:423 (1992); Daly, J. Med. Chem. 25:197 (1982).

Allosteric adenosine receptor ligands that may be used in the practice of the present invention are those set forth in U.S. Pat. No. 6,194,449 to Baraldi et al. (incorporated herein by reference), and include but are not limited to (2-amino 4,5-dimethyl-3-thienyl)-(phenyl)methanone; (2-amino-4,5-dimethyl-3-thienyl)-[(3,5-dichloro-4-amino)-phenyl)]methanone; (2-amino-3-thienyl)-(4-chlorophenyl)methanone; 2-amino-3-benzoyl-6-benzyloxycarbonyl-4,5,6,7-tetrahydrothieno[2,3-c]pyridine; 2-amino-3-benzoyl-4,5,6,7-tetrahydrothieno[2,3-c]pyridine; 2-amino-3-(4-chloro-benzoyl)-6-benzyloxycarbonyl-4,5,6,7-tetrahydrothieno[2,3-c]pyridine; 2-amino-3-(4-chloro-benzoyl)-4,5,6,7-tetrahydrothieno[2,3-c]pyridine; 2-amino-3-[8 3-(trifluoromethyl)-benzoyl]-6-(3-phenyl-prop-1-yl)-4,5,6,7-tetrahydrothieno[2,3-c]pyridine;2-amino-3-[3-(fluoromethyl)-benzoyl]-6-(phenylmethyl)-4,5,6,7-tetrahydrothieno[2,3-c] pyridine; 2-amino-3-(4-chloro-benzoyl)-6-(2-phenyleth-1-yl)-4,5,6,7-tetrahydrothieno[2,3-c]pyridine; 2-amino-3-[3-(fluoromethyl)-benzoyl]-6-(2-phenyleth-1-yl)-4,5,6,7-tetrahydrothieno[2,3-c]pyridine; 2-amino-3-(4-chloro-benzoyl)-6-(3-phenylprop-1-yl)-4,5,6,7-tetrahydrothieno[2,3-c]pyridine; 2-amino-3-(4-chloro-benzoyl)-6-(ethoxycarbonyl-methyl)-4,5,6,7-tetrahydrothieno[2,3-c]pyridine;2-amino-3-benzoyl-6-(3-methylbut-2-en-lyl)-4,5,6,7-tetrahydrothieno[2,3-c]pyridine; 2-amino-3-(4-chloro-benzoyl)-6-[4-nitro-(2-phenyleth-1-yl)]-4,5,6,7-tetrahydrothieno[2,3-c]pyridine; 2-amino-3-benzoyl-6-[4-nitro-(2-phenyleth-1-yl)]-4,5,6,7-tetrahydrothieno[2,3-c]pyridine, 2-amino-3-benzoyl-6-[2-t-butoxycarbonylamino-3-(4-hydroxyphenyl)-propion-1-yl]4,5,6,7-tetrahydrothieno[2,3-c]pyridine; 2-amino-3-benzoyl-4,5,6,7-tetrahydrobenzo[b]thiophene,4-phenyl-5,6,7,8-tetrahydro[1]benzothieno[2,3-d]pyrimidine; 2-methyl,3-ethoxycarbonyl-4-phenyl-5,6,7,8-tetrahydro[1]benzothieno[2,3-b]pyridine; 2-Amino-3-(4-bromobenzoyl)-cyclopenta[b]thiophene, 2-amino-3-benzoyl-6-(4-methylphenylsulphonyl)-4,5,6,7-tetrahydrothieno[2,3-c]pyridine, and combinations and mixtures thereof.

Additional A₁AR agonists useful in the present invention are set forth in U.S. Pat. No. 6,048,865 to Baraldi et al. (incorporated by reference) and include but are not limited to N⁶-(4-biphenyl carbonylamino)-adenosine-5′-N-ethyluronamide, N⁶-(2,4-dichlorobenzyl-carbonylamino)-adenosine-5′-N-ethyluronamide; N⁶-(4-methoxyphenyl-carbonylamino)-adenosine-5′-N-ethyluronamide; N⁶-(4-chlorophenyl-carbonylamino)-adenosine-5′-N-ethyluronamide; N⁶-(phenyl-carbonylamino)-adenosine-5′-N-ethyluronamide; N⁶-(4-sulfonamido-phenylcarbamoylamino)-adenosine-5′-N-ethyluronamide; N⁶-(4-acetyl-phenylcarbamoylamino)-adenosine-5′-N-ethyluronamide; N⁶-((R)-α-phenylethylcarbamoylamino)-adenosine-5′-N-ethyluronamide; N⁶-((S)-α-phenylethylcarbamoylamino)-adenosine-5′-N-ethyluronamide;N⁶-(5-methyl-isoxazol-3-yl-carbamoylamino)-adenosine-5′-N-ethyluronamide; N⁶-(1,3,4-thiadiazol-2-yl-carbamoylamino)-adenosine-5′-N-ethyluronamide; N⁶-bis-(4-nitrophenylcarbamoylamino)-adenosine-5′-N-ethyluronamide; N⁶-bis-(5-chloro-pyridin-2-yl-carbamoylamino)-adenosine-5′-N-ethyluronamide and combinations and mixtures thereof.

Additional ligands useful in the practice of the present invention include those set forth in, for example, U.S. Pat. Nos. 5,599,671 to Jacobson et al., 5,998,387 to Belardinelli, 5,066,655, to Olsson; 4,968,672, to Jacobson et al.; 5,298,508 to Jacobson et al., 5,248,770, to Jacobson et al.; 4,696,932 to Jacobson et al.; 5,981,524, to Peck et al.; 4,559,402 to Irikura et al.; 4,670,432 to Ward et al.; 5,773,530 to Akahane et al.; 5,565,566, to Olsson; 5,668,139 to Belardinelli et al.; 5,446,046 to Bellardinelli et al.; 5,310,916 to Jacobson et al.; and 6,040,296 to Nyce et al. (all of which are incorporated herein by reference).

Still other ligands useful in the practice of the present invention include those set forth in, for example, published PCT Applications WO 97/24363A1 to Belardinelli et al.; WO 95/11904A1 to Belardinelli et al.; WO 88/08303A1 to Olsson; WO 97137667A1 to von Lubitz et al.; WO 00/71558 to Blackburn et al.; and WO9967239A1 to Akahane et al.; European Application Nos. EP503563 to Peet et al.; EP 764647 to Connell et al.; and EP501379 to Suzuki et al.; Australian Patent Application Nos. AU1044995A1, AU699630B2, and AU728439B2, all to Belardinelli et al.; Japanese applications JP8099976A to Shiokawa et al. and JP9216883A to Kuroda; Chinese Application No. CN1206420A to Belardinelli et al. and Belgian Application No. BE 636 828 to Georges et al. (all of which are incorporated herein by reference).

Glycolipids of the present invention include, without limitation, monosialoganglioside, lactocerebroside, and galactocerebroside (in particular, NBD-galactocerebroside). Glycolipids of the present invention also include glycolipid analogs such as those set forth in U.S. Pat. No. 4,855,283 to Lockhoff et al., which is incorporated herein by reference. Such glycolipid analogs include N-glycoylamides, N-glycosylureas and N-glycosylcarbamates, each of which is substituted in the sugar residue by an amino acid. N-glycolipid analogs of the present invention also include glycosphingolipids and glycoglycerolipids. Some glycolipids have been synthesized from long chain-alkylamines and fatty acids that are linked directly with the sugars through the anomeric carbon atoms, and are also included in the present invention. Glycolipid mimics, analogs and derivatives set forth in U.S. Pat. No. 6,103,883 to Lingwood et al., U.S. Pat. No. 5,861,520 to Ogawa et al., and U.S. Pat. No. 5,589,465 to Ishida et al. (all of which are incorporated by reference herein in their entirety) are also encompassed by the present invention.

The active compounds of the present invention (e.g., A₁AR ligands and glycolipids) may alternatively and optionally be provided in the form of a free base, or in the form of a pharmaceutically acceptable salt of the active compound. The term “pharmaceutically acceptable salt” refers to a pharmaceutically acceptable salt of an A₁AR ligand or glycolipid compound, which are derived from a variety of organic and inorganic counter ions well known in the art. Such salts include, by way of example only, sodium, potassium, calcium, magnesium, ammonium, tetraalkylammonium, and the like. When the active compound contains a basic functionality, salts of organic or inorganic acids, such as hydrochloride, hydrobromide, tartrate, mesylate, acetate, maleate, oxalate and the like may be used as the pharmaceutically acceptable salt. In general, suitable pharmaceutically acceptable salts include inorganic acid addition salts such as hydrochloride, hydrobromide, sulfate, phosphate, and nitrate; organic acid addition salts such as acetate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt and N,N′-dibenzylethylenediamine salt; and salts with basic amino acid such as lysine salt and arginine salt.

The delivery of glycolipids and A₁AR ligands to a cell or a subject may occur when carrying out diagnostic methods that utilize such A₁AR ligands. Diagnostic assays utilizing ligands and A₁AR receptors are known in the art. For example, radioligand binding assays such as the binding of [³H]-DPCPX (1,3-dipropyl-8-cyclopentylxanthine) to rat brain membranes can be performed essentially according to the method previously described by Bruns et al., Proc. Natl. Acad. Sci. U.S.A., 77, 5547-5551 (1980). Other assays for binding A₁AR ligands to A₁ARs present in membranes are known. Certain assays are carried out in part by binding a monoclonal antibody to an A₁AR, e.g., when the receptor or the ligand is bound to a solid support. The present invention may be used to increase the binding of the antibody to the receptor.

Those skilled in the art will appreciate that the present invention is not limited to A₁AR receptor ligands binding to an A₁ adenosine receptor expressed in a cell or membrane. The present invention expressly includes the binding of a A₁AR receptor ligand to, for example, a partially or completely purified A₁ receptor protein, or to a polypeptide that is synthesized based on the amino acid sequence of a ligand binding site for the A₁AR protein.

In a preferred embodiment, the delivery of an A₁AR ligand is enhanced when the ligand is used in a diagnostic method set forth in U.S. patent application Ser. No. 08/748,559, filed Nov. 8, 1996 (now U.S. Pat. No. 6,159,701 to Neely, issued Dec. 12, 2000, hereinafter the '701 patent), and in U.S. patent application Ser. No. 09/569,394 to Neely, filed May 12, 2000, which is incorporated herein in its entirety by reference. The inventions set forth in this application relate in part to methods of imaging tumor cells in vivo in a subject, and comprise obtaining a sample of treatment cells from a subject, the treatment cells generally being selected from the group consisting of macrophages, monocytes and splenocytes. The treatment cells are primed by contacting the cells with a priming agent. The cells are then labeled with a radiolabeled A₁AR ligand. Finally, the labeled treatment cells are administered to the subject in an amount effective to provide a radioimage of tumor cells present in said subject. In the practice of the present invention, the affinity of the labeled A₁ adenosine receptor ligand for the receptor in the treatment cells is increased or enhanced by the presence of a glycolipid. The glycolipid may be contacted with the ligand prior to the labeling step, or contacted with the ligand concurrently with the labeling step, or contacted with the ligand after the labeling step, or delivered to the treatment cell concurrently with the A₁AR ligand, or delivered to the treatment cell prior, during, or after the labeling step, or delivered to the treatment cell prior, during or after contacting the treatment cell with the A₁AR ligand. Optionally, the glycolipid may be chemically bound (e.g., linked or conjugated) to the A₁AR ligand.

In another preferred embodiment, the present invention is carried out during the practice of the diagnostic methods set forth in U.S. patent application Ser. No. 09/569,394 to Neely, filed May 12, 2000, which is also incorporated by reference herein in its entirety. In embodiments of the invention described in that application, a sample of diagnostic cells (e.g., monocytes, macrophages, promonocytes, peripheral blood stem cells (PBSC), hematopoietic stem cells) is collected from a subject. The diagnostic cells are then tested for cytotoxicity for target cancer cells, the results of the testing providing a measure of cytotoxicity of the subject's diagnostic cells. The measure of cytotoxicity may be a measure of the number of A₁ adenosine receptors (A₁AR) in the membranes of the diagnostic cells, or a measure of the affinity of the diagnostic cells for A₁ adenosine receptor specific ligands (i.e., a measure of the affinity of the A₁ARs present in the membranes of the diagnostic cells for A₁AR specific ligands), or a measure of the ability of the diagnostic cells (i.e., the A₁ARs present in the membranes of the cells) to bind MCP-1 protein, or a measure of the ability of the diagnostic cells (i.e., the A₁ARs present in the membranes of the cells) to bind annexins. These various measurements of binding to the A₁AR may be carried out using labeled ligand binding measurement techniques known in the art, wherein binding is expressed by K_{d} (in saturation binding experiments) or Kᵢ (in competition binding experiments), with the value of Kᵢ and K_{d} being inversely related to affinity (i.e, the lower the Kᵢ or K_{d}, the higher the affinity). In general, the lower the value of the K_{d} or Kᵢ of the diagnostic cell, the greater the measure of cytotoxicity of the diagnostic cell, and the lower the risk of the subject developing cancer. In such methods, the affinity of the ligands for the receptors may be increased by the contacting of the cells/membranes with a glycolipid, or the contacting of the ligand with a glycolipid prior, during or after delivery of the ligand to the cells. Optionally, the glycolipid may be chemically bound (e.g., linked or conjugated) to the A₁AR ligand prior to, during or after contacting with the cell.

In still another preferred embodiment, the present invention is carried out during the practice of the diagnostic methods set forth in U.S. patent application Ser. No. 08/652,928, filed May 24, 1996 (now U.S. Pat. No. 5,773,306 to Neely, issued Jun. 30, 1998), which is also incorporated by reference herein in its entirety. Embodiments of the invention described in that application relate to methods and kits for the detection of endotoxin in a sample. Exemplary assays of these embodiments are competitive inhibition assays of endotoxin for the A₁AR expressed in cell membranes. A glycolipid or an analog thereof may be added to membranes expressing the A₁AR (or purified A₁AR protein or polypeptide synthesized based on the amino acid sequence of the ligand binding sites for the A₁AR) plated on the solid phase, in order to increase the stability and shelf-life of the endotoxin or A₁AR binding assay. Alternatively, treating the membranes expressing the A₁AR (or a purified A₁AR protein, or a polypeptide synthesized based on an amino acid sequence of a ligand binding site for the A₁AR) may increase the sensitivity and specificity of the endotoxin or A₁AR binding assay. The binding of endotoxin for the A₁AR competes with the binding of an A₁AR ligand for the A₁AR. The measure of binding of the ligand is negatively correlated with the amount of endotoxin present in the sample (e.g., the greater the binding of the ligand to the A₁AR, the less the amount of endotoxin present). In the practice of the present invention, the affinity of the A₁AR ligand for the receptor is increased or enhanced by the presence of a glycolipid. The glycolipid may accordingly be added to the endotoxin assay to increase the sensitivity or specificity of the assay for endotoxin. Optionally, the glycolipid may be chemically bound (e.g., linked or conjugated) to the A₁AR ligand. These methods may also be advantageously used in numerous other A₁AR binding assays that will be known or will be able to be determined by the skilled artisan.

The present invention may also be carried out in conjunction with therapeutic methods that utilize A₁AR ligands. Numerous therapies that utilize A₁AR ligands are known. For example, A₁AR ligands have been identified and implicated for use in the treatment of physiological complications resulting from cardiovascular, renal and neurological disorders. Adenosine receptor agonists have been identified for use as vasodilators (FASEB. J. 3(4), Abstract Nos. 4770 and 4773 (1989) and J. Med. Chem. 34, 2570) (1988); antihypertensive agents (D. G. Taylor et al., FASEB J. 2, 1799 (1988)); and anti-psychotic agents (T. G. Heffner et al., Psychopharmacology 98,31-38 (1989)). A₁AR agonists have been identified for use in improving renal function (R. D. Murray and P. C. Churchill, J. Pharmacol. Exp. Therap. 232,189-193 (1985)). Allosteric A₁AR or binding enhancers have shown utility in the treatment of ischemia, seizures or hypoxia of the brain. See R. F. Bruns, et al., Mol. Pharmacol. 38, 939-949 (1990) and C. A. Janusz, et al., Brain Research 567,181-187)(1991).

A₁AR ligands have additionally been found to be useful as diuretics, as bronchodilators, i.e., as antiasthmatics; in the treatment of adenosine-sensitive cardiac arrhythmias; for antinociception (i.e., as analgesics); as anticonvulsants; for cardioprotection, both short term (e.g., prior to percutaneous angioplasty (PTCA), angioplasty, and cardiac surgeries) and long term (prevention of myocardial infarction, especially in high risk patients, reduction of infarct damage, especially in high risk patients); for neuroprotection, such as stroke prevention, stroke treatment, and the treatment of epilepsy; for pain management generally, including different forms of neuropathic pain (e.g., diabetic neuropathy), post herpetic neuralgia; in anti-lipid uses such as reduction of free fatty acids, triglycerides, glucose; for adjunct therapy in diabetes, including insulin and non-insulin dependent diabetes mellitus; for stimulation of insulin secretion from the, pancreas, for increase in tissue sensitivity to insulin; for treatment of GI disorders such as diarrhea, irritable bowel disease, irritable bowel syndrome, and incontinence; for treatment of glaucoma; for treatment of sleep apnea; for treatment of cardiac disarrythmias (paroxysmal supraventricular tachycardia; for use in combination with anesthesia for post surgical pain; for treatment of inflammation; for treatment of kidney and liver disorders (e.g., in non-transplant patients suffering from liver failure, pre-transplant patients, or for transplant patients having hepato-renal syndrome); for treatment of sepsis, septicemia, endotoxemia, endotoxin-induced organ/tissue injury, and ischemia-reperfusion organ/tissue injury; for treatment and prevention of fibrosis and sclerosis; for treatment of AIDS and other immune disorders; and for treatment of tumors and cancers.

Preferred embodiments of the present invention are carried out during the practice of the methods set forth in the '701 patent and in U.S. patent Ser. No. 09/224,534, filed Dec. 31, 1998 (now U.S. Pat. No. 6,117,445 to Neely, issued Sep. 12, 2000, hereinafter the '445 patent), which are incorporated herein in their entirety by reference. In addition to relating to methods of imaging tumors, as set forth above, the '701 patent also relates to methods of treating tumors, and particularly to methods of treating tumors using A₁ adenosine-receptor activated cells, such as monocytes, macrophages and/or splenocytes. Activation of these cells may be carried out by activating treatment cells by contacting them with an A₁ adenosine receptor agonist to induce cytotoxicity in the cells, and then administering the cytotoxic treatment cells to the subject. In embodiments of the present invention, the affinity of the binding of the agonist to the A₁AR may be increased by the co-administration of a glycolipid with the A₁AR ligand. When carrying out the present invention in conjunction with the '701 patent, the affinity of the ligands for the receptors may be increased by contacting the cells/membranes with a glycolipid, or contacting the ligand with a glycolipid prior, during or after delivery of the ligand to the cells. Optionally, the glycolipid may be chemically bound (e.g., linked or conjugated) to the A₁AR ligand prior to, during or after contacting with the cells.

In another preferred embodiment, the present invention is carried out during the practice of the methods set forth the '445 patent. The methods of this application relate to methods of treating fibrosis and sclerosis using A₁ adenosine receptor antagonists. One embodiment of the invention relates to methods of treating fibrosis or sclerosis in a subject in need of such treatment by administering a composition containing an A₁ adenosine receptor antagonist, a P_{2X} purinoceptor antagonist, or a combination of at least one A₁ adenosine receptor antagonist and at least one P_{2X} purinoceptor antagonist. In embodiments of the present invention, the affinity of the binding of an A₁AR ligand (i.e., an A₁AR antagonist) to the A₁AR is increased by the co-administration of a glycolipid with the A₁AR ligand. When carrying out the present invention in conjunction with the '445 patent, the affinity of the ligands for the receptors may be increased by contacting the tissue(s) of the subject with a glycolipid prior, during or after delivery of the ligand to the tissue(s) of the subject. The affinity of the ligands for the receptors may also be increased by contacting the ligand with a glycolipid prior, during or after delivery of the ligand to the tissue(s) of the subject. Optionally, the glycolipid may be chemically bound (e.g., linked or conjugated) to the A₁AR ligand prior to, during or after contacting the ligand with the targeted tissue of the subject.

The present invention also provides pharmaceutical formulations, both for veterinary and for human medical use, which comprise an A₁AR adenosine receptor ligand and a glycolipid formulated together with one or more pharmaceutically acceptable carriers, and optionally any other therapeutic ingredients.

The amount or dosage of any specific active compound, the use of which is in the scope of the present invention, will vary somewhat from compound to compound, application to application, and will depend, among other things, upon the condition of the subject or cell and the route of delivery. When being delivered in a therapeutic setting, the duration of the treatment can be determined by the medical practitioner. A daily dose can be administered either by a single dose in the form of an individual dosage unit or several smaller dosage units, or by multiple administration of subdivided dosages at certain intervals. In another embodiment, the formulation is delivered as a continuous infusion (e.g., intravenously). The carrier(s) must be pharmaceutically acceptable in the sense of being compatible with the other ingredients of the formulation and not unduly deleterious to the recipient thereof.

When used in therapeutic settings, the formulations may be suitable for inhalational (e.g., as an aerosol), oral, rectal, topical, nasal, ophthalmic, parenteral (including but not limited to subcutaneous, intramuscular, intravenous, and intraarterial), intraarticular, intrapleural, intraperitoneal, vaginal, bladder instillation, and intracerebral (alternatively, into the cerebral spinal space) administration. Formulations suitable for oral, inhalational, and parenteral administration are preferred.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier, a finely divided solid carrier, or both, and then, if necessary, shaping the product into desired formulations.

Formulations of the present invention suitable for oral administration may be presented as discrete units such as capsules, cachets, tablets or lozenges, each containing a predetermined amount of the integrase inhibiting agent as a powder or granules; or a suspension in an aqueous liquid or non-aqueous liquid such as a syrup, an elixir, an emulsion or a draught.

Formulations suitable for parenteral administration conveniently comprise a sterile aqueous preparation of the active compound, which is preferably isotonic with the blood of the recipient and pyrogen-free.

The present invention provides pharmaceutical formulations suitable for intravenous or intramuscular injection. If a solution is desired, water is a preferred carrier with respect to water-soluble compounds or salts. An organic vehicle, such as glycerol, propylene glycol, polyethylene glycol, or mixtures thereof, may also be suitable. The solution in either instance may then be sterilized in any suitable manner, for example, by filtration. Subsequent to sterilization, the solution may be filled into appropriate receptacles, such as depyrogenated glass vials. The filling is preferably done by an aseptic method. Sterilized closures may then be placed on the vials and, if desired, the vial contents may be lyophilized.

In addition to the ligands and glycolipid of the present invention, the pharmaceutical formulation may contain other additives, such as pH adjusting additives. Useful pH adjusting agents include but are not limited to acids, bases or buffers, such a sodium lactate, sodium acetate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical formulations of the present invention may be lyophilized using techniques well known in the art.

In yet another aspect of the present invention, there is provided an injectable, stable, sterile composition comprising a formulation of the present invention, or a salt thereof, in a unit dosage form in a sealed container. The formulation is provided in the form of a lyophilizate which is capable of being reconstituted with a suitable pharmaceutically acceptable carrier to form a liquid composition suitable for injection thereof into the subject. When the compound or salt is substantially water-insoluble, a sufficient amount of emulsifying agent which is physiologically acceptable may be employed in sufficient quantity to emulsify the compound or salt in an aqueous carrier. One useful emulsifying agent is phosphatidylcholine.

Further, the present invention provides liposomal formulations comprising the ligands of the present invention together with a glycolipid or analog thereof. The technology for forming liposomal suspensions is well known in the art. The formulation may be incorporated into lipid vesicles using conventional liposome technology. The lipid layer employed may be of any conventional composition and may contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes that are produced may be reduced in size, as through the use of standard sonication and homogenization techniques.

The liposomal formulations containing the pharmaceutical formulation may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the active compounds or a plurality of solid particles of the active compounds. The desired formulation may be placed in a small chamber and nebulized. Nebulization may be accomplished by compressed air or by ultrasonic energy to form a plurality of liquid droplets or solid particles comprising the compounds of salts. The solid particles can be obtained by processing the formulation, in any appropriate manner known in the art, such as by micronization. Commercial nebulizers are available to achieve this purpose.

The present invention provides advantages over presently known methods of delivering or administrating A₁AR ligands in that the administration or delivery of a glycolipid increases the affinity of the ligand for the A₁AR. In certain embodiments of the invention, the administration or delivery of the glycolipid increases the efficiency of the delivery of the drug to the cell, tissue or subject. This effect provides for increased uptake of the ligands across, for example, the blood-brain barrier, and the tissue barriers of, for example, liver, gut, skin, vagina, mucosa of the respiratory tract (including but not limited to mucosa of the nose, mouth, trachea, and bronchi) and brain.

In certain embodiments of the invention, the administration or delivery of the glycolipid with the ligand, or delivery or administration of the glycolipid to the target cell, receptor protein, membrane, tissue or subject, increases the bioavailability of the ligand. In other embodiments of the invention, the administration or delivery of the glycolipid with the ligand, or delivery or administration of the glycolipid to the target cell, receptor protein, membrane, tissue or subject, increases the solubility of the ligand. In still other embodiments of the invention, the combination or conjugation of the glycolipid with the ligand allows for an alteration in the solubility of the ligand (for example, an alteration from a water-soluble ligand to a non-water soluble ligand). Increasing bioavailability and solubility would generally decrease the amount of ligand that would have to be administered to the subject or cell.

The following Examples are provided to illustrate the present invention, and should not be construed as limiting thereof.

### EXAMPLE 1

### Human Pulmonary Arterial Endothelial Cells (Human PAECs)

### Culture and Membrane Preparation

Human PAECs were obtained from BioWhittaker Inc. (Walkersville, Md.) and grown in a multilayer tissue culture vessel for membrane preparation and in 24-well culture plates for functional studies in an atmosphere of 95% O₂ and 5% CO₂. The cells were grown and maintained in medium recommended by the manufacturer. The cells were washed 3 times with PBS and then suspended in lysis buffer (10 mM Tris HCl pH 7.4, 5 mM EDTA, 10 μg/ml soybean trypsin inhibitor, 10 μg/ml benzamidine, 2 μg/ml pepstatin). The cells were homogenized by sonication. The homogenate was centrifuged at 1000×g at 4° C. for 10 minutes. The supernatant was centrifuged at 30000×g for 30 minutes. The pellet was reconstituted in reconstitution buffer (50 mM Tris HCl pH 7.4, 5 mM EDTA, 10 mM MgCl₂, 10 μg/ml soybean trypsin inhibitor, 10 μg/ml benzamidine, 2 μg/ml pepstatin). The protein content was determined by Bradford reagent using bovine serum albumin as standard. The aliquots were stored at −80° C. until used.

### EXAMPLE 2

### Radioligand Competition Binding Experiments

Radioligand competition binding experiments were performed with membranes from human PAECs in a total volume of 0.2 ml in 50 mM Tris HCl buffer, adenosine deaminase 0.2 U/ml, pH 7.4 at room temperature with the selective A₁ adenosine receptor antagonist radioligand [¹²⁵I] BWA844U (0.4 nM), the highly selective A₁ adenosine receptor agonist radioligand [³H] 2-chloro, N⁶-cyclopentyladenosine (CCPA) (0.4 nM), or the highly selective A₂ₐ adenosine receptor agonist radioligand [³H] CGS 21680 (2 nM). Non-specific binding was determined in presence of N⁶—R-phenylisopropyladenosine (R—PIA) (100 μM) for [¹²⁵I] BWA844U and [³H] CCPA or 5′-(N-ethylcarboxamido)-adenosine (NECA) (100 μM) for [³H] CGS 21680. For [¹²⁵I] BWA844U experiments, the incubation was terminated after 2 hours by filtration over GF/C filters using a cell harvester. The filter bound radioactivity was counted in a gamma counter (CliniGamma, LKB). For [³H] CCPA and [³H] CGS 21680 experiments, the incubation was terminated after 2 hours by filtration over GF/C filters using a cell harvester. The filter bound radioactivity was counted in a liquid scintillation counter. The following adenosine receptor agonists and lipopolysaccharides were tested in these radioligand competition binding experiments: highly selective A₁ adenosine receptor agonist, CCPA (0.037 pg/ml-3.7 μg/ml), selective A₂ adenosine receptor agonist 2-phenylaminoadenosine (CV 1808) (0.036 pg/ml-3.6 μg/ml), Escherichia coli LPS (0.1 pg/ml-10 μg/ml), Salmonella typhimurium LPS (0.1 pg/ml-10 μg/ml), Klebsiella pneumoniae LPS (0.1 pg/ml-10 μg/ml), and Pseudomonas aeruginosa LPS (0.1 pg/ml-10 μg/ml). To test for selectivity, enterotoxin (1 pg/ml-10 μg/ml), diphosphoryl lipid A (1 pg/ml-100 μg/ml), monosialoganglioside (1 pg/ml-100 μg/ml), lactocerebroside (1 pg/ml-100 μg/ml), and NBD-galactocerebroside (1 pg/ml-100 μg/ml) were tested. Stock solutions of lactocerebroside, NBD-galactocerebroside and diphosphoryl lipid A were prepared in DMSO at a concentration of 1 mg/ml and further dilutions of these ligands were made in the buffer with a final concentration of DMSO≦10%. All other reagents were dissolved in 50 mM Tris HCl buffer, pH 7.4. Three experiments were performed on separate days for each LPS, adenosine receptor agonist, enterotoxin, diphosphoryl lipid A, or glycolipid and assayed in duplicate.

### EXAMPLE 3

### Statistical Analysis

Radioligand binding data was analyzed by nonlinear regression analysis using GraphPad Prism (version 3.0). This program was used to plot the data and calculate the IC₅₀ values. The IC₅₀ values are calculated using the following formula:

$$Y = Bottom + \frac{( Top - Bottom )}{1 + {10}^{X - Log IC 50}}$$

where

Y=Binding at any given conc.

Top=Binding in absence of any competing ligand

Bottom=Binding in presence of highest conc. of the competing ligand

X=Log Conc. of the competing ligand

### EXAMPLE 4

### Radioligand Competition Binding Experiments

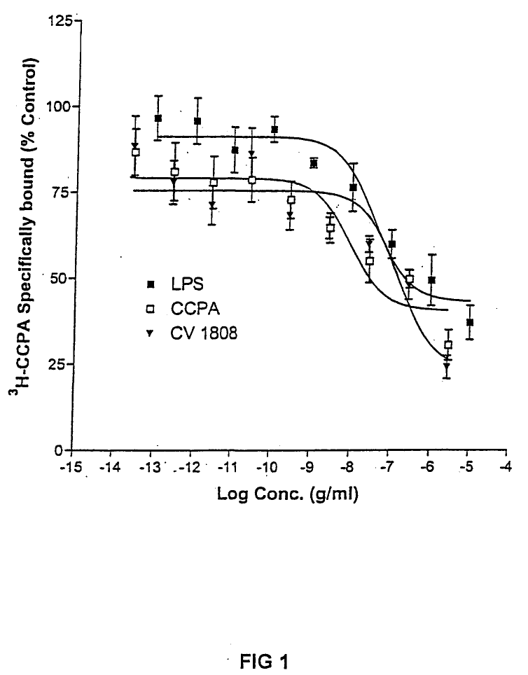

In radioligand competition binding experiments, with the highly selective A₁ adenosine receptor agonist radioligand, [³H] CCPA, in membranes from human PAECs, the selective A₁ adenosine receptor agonist, CCPA (0.037 pg/ml-3.7 μg/ml), the selective A₂ adenosine receptor agonist, CV 1808 (0.036 pg/ml-3.6 μg/ml), and Escherichia coli LPS (0.1 pg/ml-10 μg/ml) displace binding of [³H] CCPA in a competitive, dose dependent manner (FIG. 1). Based on the calculated [IC₅₀s, the agonist potency profile is CCPA>LPS>CV 1808. The calculated IC₅₀s of CCPA, LPS, and CV 1808 are 9.4 ng/ml, 111 ng/ml, and 155 ng/ml, respectively.

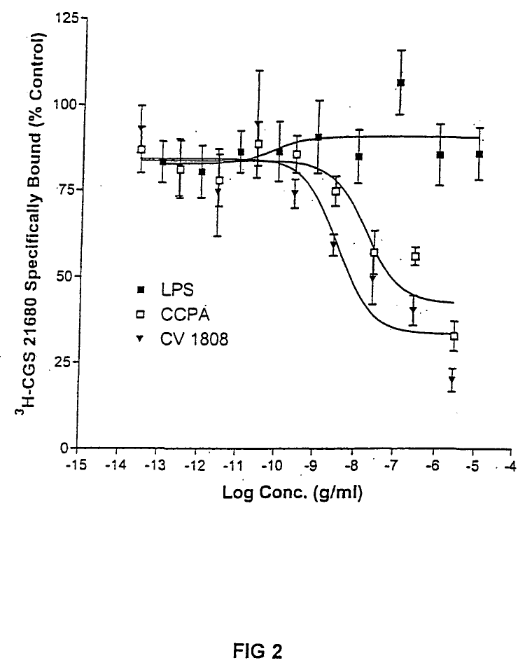

In radioligand binding competition experiments with the highly selective A₂ₐ adenosine receptor agonist radioligand, [³H] CGS 21680, in membranes from human PAECs, CV 1808 (0.036 pg/ml-3.6 μg/ml) and CCPA (0.037 pg/ml-3.7 μg/ml) displace binding of [³H] CGS 21680 in a competitive, dose dependent manner (FIG. 2). The calculated IC₅₀s for CV 1808 and CCPA are 3.5 ng/ml and 17.8 ng/ml, respectively. There is no displacement of [³H] CGS 21680 binding by Escherichia coli LPS (0.1 pg/ml-10 μg/ml) (FIG. 2).

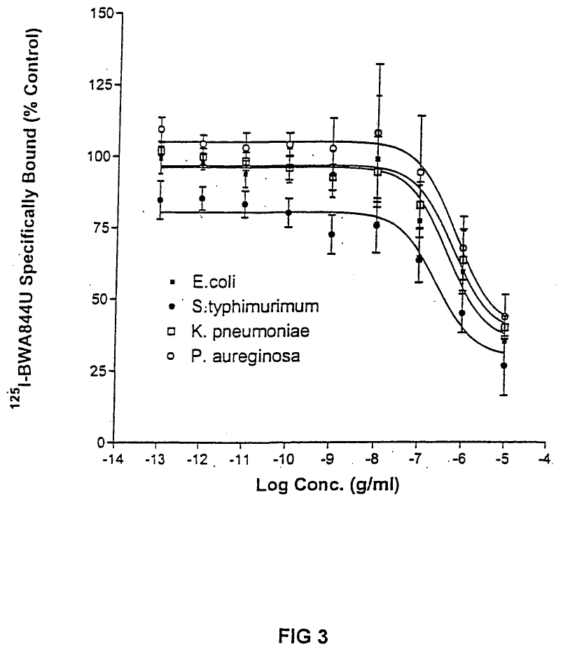

In radioligand binding competition experiments with the selective A₁ adenosine receptor antagonist radioligand, [¹²⁵I] BWA844U, in membranes from human PAECs, LPSs from Escherichia coli (E. coli) LPS (0.1 pg/ml-10 μg/ml), Salmonella typhimurium (S. typhimurium) LPS (0.1 pg/ml-10 μg/ml), Klebsiella pneumoniae (K. pneumoniae) LPS (0.1 pg/ml-10 μg/ml), and Pseudomonas aeruginosa (P. aeruginosa) LPS (0.1 pg/ml-10 μg/ml) displace binding of [¹²⁵I] BWA844U in a competitive, dose dependent manner (FIG. 3). The calculated IC₅₀s of E. coli LPS, S. typhimurium LPS, K. pneumoniae LPS, and P. aeruginosa LPS are 195 ng/ml, 290 ng/ml, 602 ng/ml, and 693 ng/ml, respectively. There was no displacement of [¹²⁵I] BWA844U binding by enterotoxin (≦10 μg/ml), diphosphoryl lipid A (≦10 μg/ml), monosialoganglioside (≦1 μg/ml), lactocerebroside (≦100 μg/ml), and NBD-galactocerebroside (≦100 μg/ml) (data not shown). Diphosphoryl lipid A (100 μg/ml) displaces [¹²⁵I] BWA844U binding in human PAECs approximately 50% (data not shown). Monosialoganglioside (10 and 100 μg/ml) displaces [¹²⁵I] BWA844U binding in human PAECs approximately 25% and 50%, respectively (data not shown). DMSO (10%) had no effect on total or nonspecific binding of [¹²⁵I] BWA844U.

### EXAMPLE 5

### Effect of Glycolipids on A₁AR Ligand Binding

Lactocerebroside (1.0 pg/ml to 1.0 μg/ml) has no effect on [¹²⁵I] BWA844U binding in human PAECs. Lactocerebroside (10 μg/ml) and lactocerebroside (100 μg/ml) increase [¹²⁵I] BWA844U binding in human PAECs approximately 20% and 50%, respectively.

NBD-Galactocerebroside (1.0 pg/ml to 10.0 μg/ml) has no effect on [¹²⁵I] BWA844U binding in human PAECs. NBD-Galactocerebroside (100 μg/ml) increases [¹²⁵I] BWA844U binding in human PAECs approximately 75%.

The foregoing is illustrative of the present invention and is not to be construed as limiting thereof. The invention is defined by the following claims, with equivalents of the claims to be included therein.

That which is claimed:

1. A method of increasing the affinity of an A₁ adenosine receptor (A₁AR) ligand for an A₁ adenosine receptor comprising:
contacting an A₁ adenosine receptor ligand with a glycolipid or an analog thereof; and
binding the contacted A₁ adenosine receptor ligand with an A₁ adenosine receptor.

2. The method according to claim 1, wherein the A₁ adenosine receptor ligand is an A₁ adenosine receptor antagonist.

3. The method according to claim 1, wherein the A₁ adenosine receptor ligand is an A₁ adenosine receptor agonist.

4. The method according to claim 1, wherein the A₁ adenosine receptor ligand is an antibody specific for the A₁ adenosine receptor.

5. The method according to claim 4, wherein the antibody is a monoclonal antibody.

6. The method according to claim 1, wherein the A₁ adenosine receptor ligand is an endotoxin.

7. The method according to claim 6, wherein the endotoxin is lipopolysaccharide (LPS)

8. The method according to claim 1, wherein the glycolipid is selected from the group consisting of monosialoganglioside, lactocerebroside, and galactocerebroside, NBD-galactocerebroside, and mixtures thereof.

9. The method according to claim 1, wherein the A₁ adenosine receptor is in a membrane.

10. The method according to claim 1, wherein the A₁ adenosine receptor is purified A₁ adenosine receptor protein.

11. The method according to claim 1, wherein the A₁ adenosine receptor is a polypeptide that is synthesized based on an amino acid sequence of a ligand binding site for the A₁AR protein.

12. The method according to claim 1, wherein the glycolipid or analog thereof is chemically linked to the A₁ adenosine receptor ligand.

13. The method according to claim 1, wherein the glycolipid or analog thereof is conjugated to the A₁ adenosine receptor ligand.

14. The method according to claim 1, wherein the glycolipid or analog thereof is formulated in a liposome with the A₁ adenosine receptor ligand.

15. A method of increasing the affinity of an A₁ adenosine receptor (A₁AR) ligand for an A₁ adenosine receptor comprising:
contacting an A₁ adenosine receptor with a glycolipid or an analog thereof; and
binding the contacted A₁ adenosine receptor with an A₁ adenosine receptor ligand.

16. The method according to claim 15, wherein the A₁ adenosine receptor ligand is an A₁ adenosine receptor antagonist.

17. The method according to claim 15, wherein the A₁ adenosine receptor ligand is an A₁ adenosine receptor agonist.

18. The method according to claim 15, wherein the A₁ adenosine receptor ligand is an antibody specific for the A₁ adenosine receptor.

19. The method according to claim 18, wherein the antibody is a monoclonal antibody.

20. The method according to claim 15, wherein the A₁ adenosine receptor ligand is an endotoxin.

21. The method according to claim 20, wherein the endotoxin is lipopolysaccharide (LPS).

22. The method according to claim 15, wherein the glycolipid is selected from the group consisting of monosialoganglioside, lactocerebroside, and galactocerebroside, NBD-galactocerebroside, and mixtures thereof.

23. The method according to claim 15, wherein the A₁ adenosine receptor is in a membrane.

24. The method according to claim 15, wherein the A₁ adenosine receptor is purified A₁ adenosine receptor protein.

25. The method according to claim 15, wherein the A₁ adenosine receptor is a polypeptide that is synthesized based on an amino acid sequence of a ligand binding site for the A₁AR protein.

26. The method according to claim 15, wherein the glycolipid or analog thereof is chemically linked to the A₁ adenosine receptor ligand.

27. The method according to claim 15, wherein the glycolipid or analog thereof is conjugated to the A₁ adenosine receptor ligand.

28. The method according to claim 15, wherein the glycolipid or analog thereof is formulated in a liposome with the A₁ adenosine receptor ligand.

29. A method of increasing the affinity of an A₁ adenosine receptor ligand for an A₁ adenosine receptor, comprising concurrently contacting an A₁ adenosine receptor, an A₁ adenosine receptor ligand, and a glycolipid or analog thereof.

30. The method according to claim 29, wherein the A₁ adenosine receptor ligand is an A₁ adenosine receptor antagonist.

31. The method according to claim 29, wherein the A₁ adenosine receptor ligand is an A₁ adenosine receptor agonist.

32. The method according to claim 29, wherein the A₁ adenosine receptor ligand is an antibody specific for the A₁ adenosine receptor.

33. The method according to claim 32, wherein the antibody is a monoclonal antibody.

34. The method according to claim 29, wherein the A₁ adenosine receptor ligand is an endotoxin.

35. The method according to claim 34, wherein the endotoxin is lipopolysaccharide (LPS).

36. The method according to claim 29, wherein the glycolipid is selected from the group consisting of monosialoganglioside, lactocerebroside, and galactocerebroside, NBD-galactocerebroside, and mixtures thereof.

37. The method according to claim 29, wherein the A₁ adenosine receptor is in a membrane.

38. The method according to claim 29, wherein the A₁ adenosine receptor is purified A₁ adenosine receptor protein.

39. The method according to claim 29, wherein the A₁ adenosine receptor is a polypeptide that is synthesized based on an amino acid sequence of a ligand binding site for the A₁AR protein.

40. The method according to claim 29, wherein the glycolipid or analog thereof is chemically linked to the A₁ adenosine receptor ligand.

41. The method according to claim 29, wherein the glycolipid or analog thereof is conjugated to the A₁ adenosine receptor ligand.

42. The method according to claim 29, wherein the glycolipid or analog thereof is formulated in a liposome with the A₁ adenosine receptor ligand.

43. In a method of delivering an A₁ adenosine receptor ligand to an A₁ adenosine receptor for the purpose of carrying out a diagnostic test, the improvement consisting of increasing the affinity of an A₁ adenosine receptor ligand for the A₁ adenosine receptor by:
contacting an A₁ adenosine receptor ligand with a glycolipid or an analog thereof; and
binding the contacted A₁ adenosine receptor ligand with an A₁ adenosine receptor.

44. The method of claim 43, wherein the A₁ adenosine receptor is present in a cell or cell membrane.

45. The method of claim 43, wherein the A₁ adenosine receptor is purified A₁ adenosine receptor protein.

46. The method of claim 43, wherein the contacting step comprises chemically linking the A₁AR ligand and the glycoplipid or analog thereof.

47. In a method of delivering an A₁ adenosine receptor ligand to an A₁ adenosine receptor for the purpose of carrying out a diagnostic test, the improvement consisting of increasing the affinity of an A₁ adenosine receptor ligand for the A₁ adenosine receptor by:
contacting an A₁ adenosine receptor with a glycolipid or an analog thereof; and
binding the contacted A₁ adenosine receptor with an A₁ adenosine receptor ligand.

48. The method of claim 47, wherein the A₁ adenosine receptor is in a cell or cell membrane.

49. The method of claim 47, wherein the A₁ adenosine receptor is purified A₁ adenosine receptor protein.

50. In a method of administering an A₁AR ligand to a subject in need of such treatment, the improvement consisting of increasing the affinity of an A₁AR ligand for the A₁AR by administering to the subject the A₁AR ligand with a glycolipid or an analog thereof.

51. The method of claim 50, wherein the contacting step comprises chemically linking the A₁AR ligand and the glycolipid or analog thereof.

52. In a method of administering an A₁AR ligand to a subject in need of such treatment, the improvement consisting of increasing the affinity of an A₁AR ligand for the A₁AR by administering to the subject a glycolipid or an analog thereof and administering an A₁AR ligand.

53. A pharmaceutical formulation comprising:
an A₁ adenosine receptor ligand;
a glycolipid or glycolipid analog in an amount sufficient to enhance binding of the A₁ adenosine receptor ligand for the A₁ adenosine receptor; and
a pharmaceutically acceptable carrier.

54. The formulation of claim 53, wherein the A₁ adenosine receptor ligand is an A₁ adenosine receptor antagonist.

55. The formulation of claim 53, wherein the A₁ adenosine receptor ligand is an A₁ adenosine receptor agonist.

56. The method of claim 53, wherein the formulation is a liposomal formulation.

57. A method of increasing the affinity of an A₁ adenosine receptor (A₁AR) ligand for a binding site polypeptide of an A₁ adenosine receptor comprising:
contacting an A₁ adenosine receptor ligand with a glycolipid or an analog thereof; and
binding the contacted A₁ adenosine receptor ligand with a binding site polypeptide of an A₁ adenosine receptor.

58. The method of claim 57, wherein the binding site polypeptide is a polypeptide that is synthesized based on an amino acid sequence of a ligand binding site for the A₁AR protein.

59. The method of claim 57, wherein the binding site polypeptide is a purified binding site polypeptide.

60. A method of increasing the affinity of an A₁ adenosine receptor (A₁AR) ligand for a binding site polypeptide of an A₁ adenosine receptor comprising:
contacting a binding site polypeptide of an A₁ adenosine receptor with a glycolipid or an analog thereof; and
binding the contacted binding site polypeptide of an A₁ adenosine receptor with an A₁ adenosine receptor ligand.

61. The method of claim 60, wherein the binding site polypeptide is a polypeptide that is synthesized based on an amino acid sequence of a ligand binding site for the A₁AR protein.

62. The method of claim 60, wherein the binding site polypeptide is a purified binding site polypeptide.
